(19) United States
(12) Patent Application Publication
(10) Pub. No.: US 2010/0185056 A1
(43) Pub. Date: Jul. 22, 2010

(54) SYSTEM FOR ADVANCING IN A BODY LUMEN

(76) Inventors: Tal Gordon, Hod HaSharon (IL); Izhak Fabian, Kfar Trumam (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 12/665,100

(22) PCT Filed: Jun. 22, 2008
(86) PCT No.: PCT/IL08/00848

§ 371 Date: Dec. 17, 2009

Related U.S. Application Data
(60) Provisional application No. 60/945,354, filed on Jun. 21, 2007.

(51) Int. Cl. A61B 1/12

(52) U.S. Cl. 600/158

(57) ABSTRACT

A system for advancing in a body lumen including a guide member insertable into a body lumen, apparatus for providing a forward propulsion force on the guide member to advance the guide member distally into the body lumen, and a lumen formed through the guide member connected to a fluid source, wherein fluid flowing through and out of the lumen cleans the body lumen as the forward propulsion force propels the guide member distally in the body lumen.

### FIELD OF THE INVENTION

The present invention relates generally to systems for advancing in body lumens, such as the gastrointestinal (GI) tract, and particularly to a system that advances through the body lumen by means of percussion forces.

### BACKGROUND OF THE INVENTION

Many imaging devices are known for producing medical images of body lumens, such as the gastrointestinal (GI) tract. For example, endoscopy is widely used for observing, photographing tissue, and taking specimens from lesions and the like. In a conventional method of examining a colon using an endoscope, for example, the endoscope is typically manually inserted into the colon. In this manual technique, patients may often complain of abdominal pain and distention because the colon is extended or excessively dilated, thereby complicating the endoscopic procedure. There may be a risk for the colon to bleed and be accidentally perforated. Insertion of an endoscope through the sigmoid colon and into the descending colon, or through the splenic flexure, the transverse colon, the hepatic flexure or parts affected by previous operations may also be accompanied with difficulty.

### SUMMARY OF THE INVENTION

The present invention seeks to provide an improved system for advancing in body lumens, such as the gastrointestinal (GI) tract, as is described more in detail hereinbelow. The invention is described hereinbelow with reference to the GI tract, but it is understood that the invention is not limited to the GI tract and may be used for other body lumens as well. Unlike the prior art, which may inflate and anchor balloons and similar devices to the GI tract wall in an attempt to overcome the slippery and unsteady GI tract, the present invention navigates through the GI tract by means of percussion regardless of the environmental conditions of the GI tract. The navigation or motion may be assisted not only by percussion but also by manual forces applied by the practitioner, and/or propulsion forces caused by vibrations, water jets pulsation, or any combination thereof.

There is thus provided in accordance with an embodiment of the present invention a system for navigating a body lumen including a guide member insertable into a body lumen, apparatus for providing a forward propulsion force on the guide member to advance the guide member distally into the body lumen, and a lumen formed through the guide member connected to a fluid source, wherein fluid flowing through and out of the lumen cleans the body lumen as the forward propulsion force propels the guide member distally in the body lumen.

An imaging assembly may be disposed in a distal portion of the guide member adapted to create images of internal portions of the body lumen. Other sensing devices may also be disposed in the guide member, such as but not limited to, temperature sensors, pH sensors, chemical sensors, etc. A drain tube may be provided through which waste material from the fluid flowing in the body lumen is drained out of the body lumen.

In accordance with an embodiment of the present invention the forward propulsion force together with the fluid breaks down and dissolves waste material as the guide member advances through the body lumen.

The apparatus for providing the forward propulsion force may include at least one percussion element disposed in a first channel formed in the guide member, the first channel having a distal terminal end, wherein the at least one percussion element is arranged to be forced distally along the first channel to strike the distal terminal end, thereby causing the guide member to advance distally in the body lumen. The apparatus for providing the forward propulsion force may include a vibratory element that sets up vibrations in the guide member. The apparatus for providing the forward propulsion force may include a jet pulsation device that creates jet pulses of fluid to propel the guide member.

The imaging assembly may be disposed in a swivel head pivotally mounted on a distal end of the guide member. A lumen may be formed through the swivel head for passing therethrough devices. The head may be swiveled and manipulated about a joint by means of a manipulator rod disposed in a third channel formed in the guide member. A second channel may be formed in the guide member in fluid communication with the first channel, the second channel being used to impart a force on the at least one percussion element that moves it proximally along the first channel away from the distal terminal end.

The guide member may include multiple percussion elements that are limited to travel in discrete regions of the first channel, wherein at each discrete region, the percussion elements are arranged to strike either a distal terminal end or a proximal stop formed in the first channel for each discrete region.

There is also provided in accordance with an embodiment of the present invention system for navigating and imaging a body lumen including a guide member insertable into a body lumen, an imaging assembly disposed in a distal portion of the guide member adapted to create images of internal portions of the body lumen, at least one percussion element disposed in a first channel formed in the guide member, the first channel having a distal terminal end, and a force applicator arranged to apply a force on the at least one percussion element to propel the at least one percussion element distally along the first channel and strike the distal terminal end, thereby causing the guide member to advance distally in the body lumen.

### BRIEF DESCRIPTION OF THE DRAWINGS

The present invention will be understood and appreciated more fully from the following detailed description taken in conjunction with the drawings in which:

FIG. 1 is a simplified pictorial illustration of a system for navigating and imaging body lumens, constructed and operative in accordance with an embodiment of the present invention;

FIG. 2A is a more detailed illustration of a guide member of the system of FIG. 1, showing internal components, in accordance with an embodiment of the present invention; FIG. 2B is a head-on view of the guide member of FIG. 2A; FIG. 2C is a sectional illustration of the guide member of FIG. 2A, taken along lines 2C-2C in FIG. 2A; FIG. 2D is a simplified pictorial illustration of the guide member of the system of FIG. 1 in a different view, showing a lumen for passing therethrough tools and the like; FIG. 2E is a simplified pictorial illustration of the guide member of the system of FIG. 1 with multiple percussion elements that are limited to travel in discrete regions of a channel in the guide member of the system;

FIG. 3 is a simplified, close-up illustration of an introducer which may be used to introduce the guide member into the body lumen, and a drain tube useful for draining waste material and the like from the body lumen, in accordance with another embodiment of the present invention;

FIG. 4 is a simplified schematic illustration of a force applicator used to propel and retract the guide member of the system of the invention, in accordance with another embodiment of the present invention; and

FIG. 5 is a simplified graphical illustration of a sequence of displacements of the percussion element due to force/pressure applied thereupon by the force applicator, in accordance with another embodiment of the present invention.

### DETAILED DESCRIPTION OF EMBODIMENTS

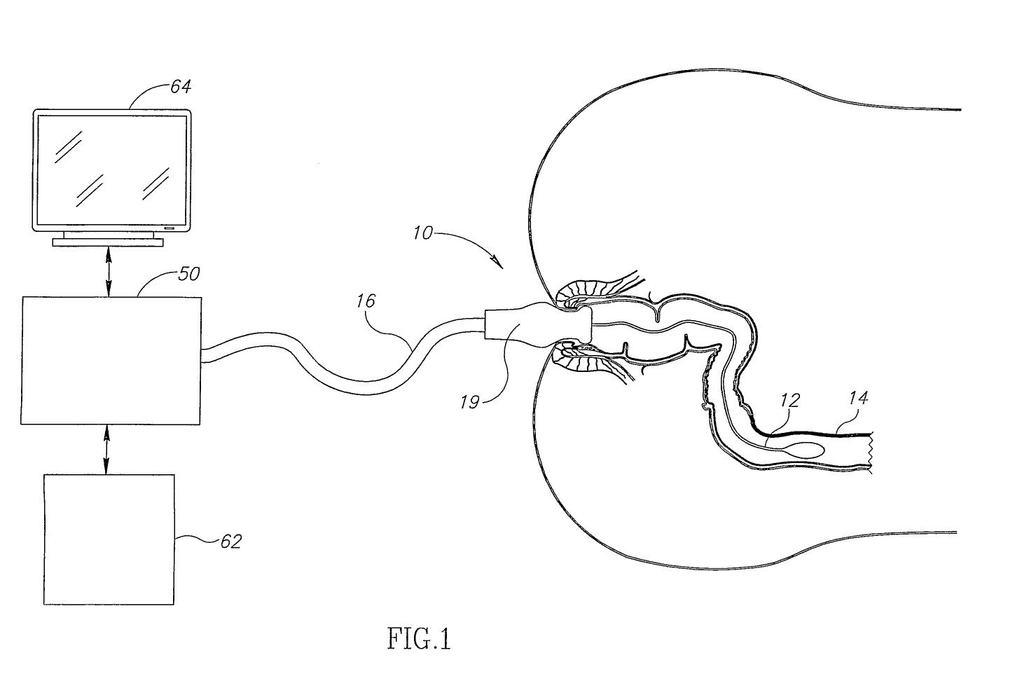

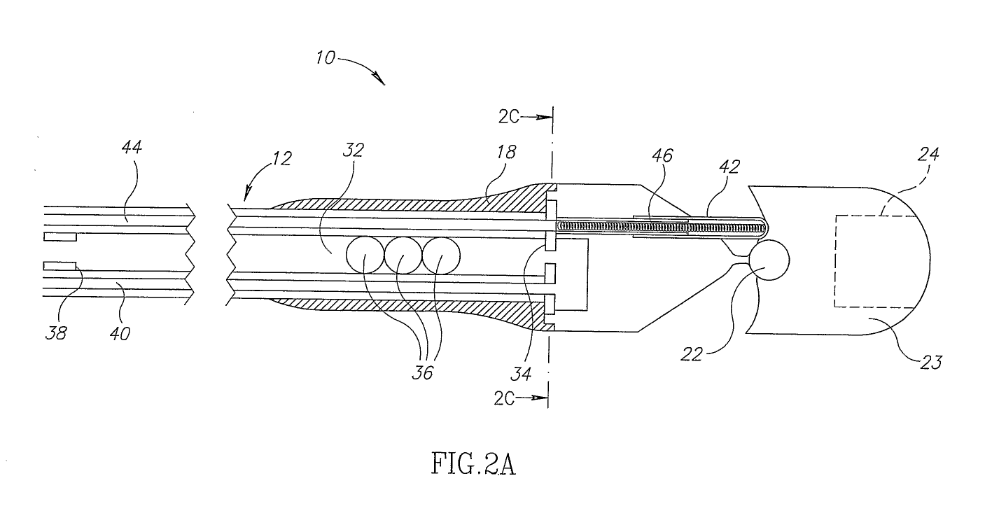

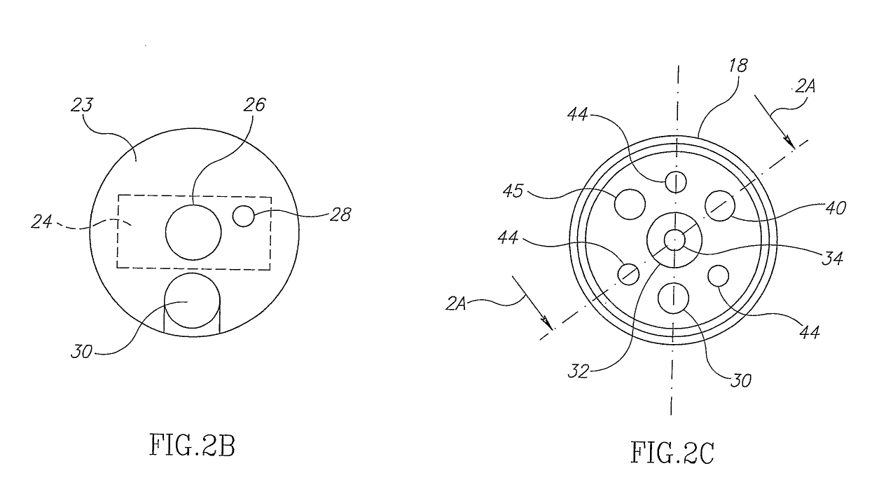

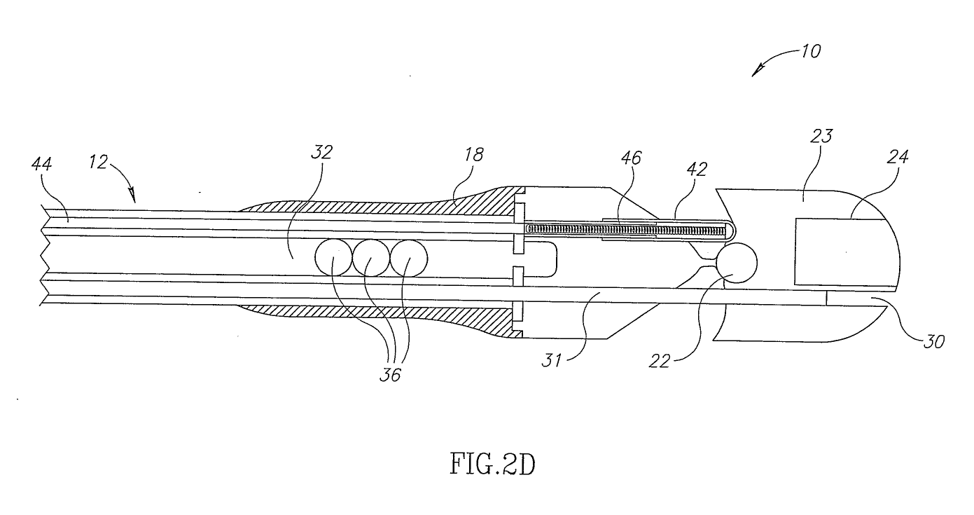

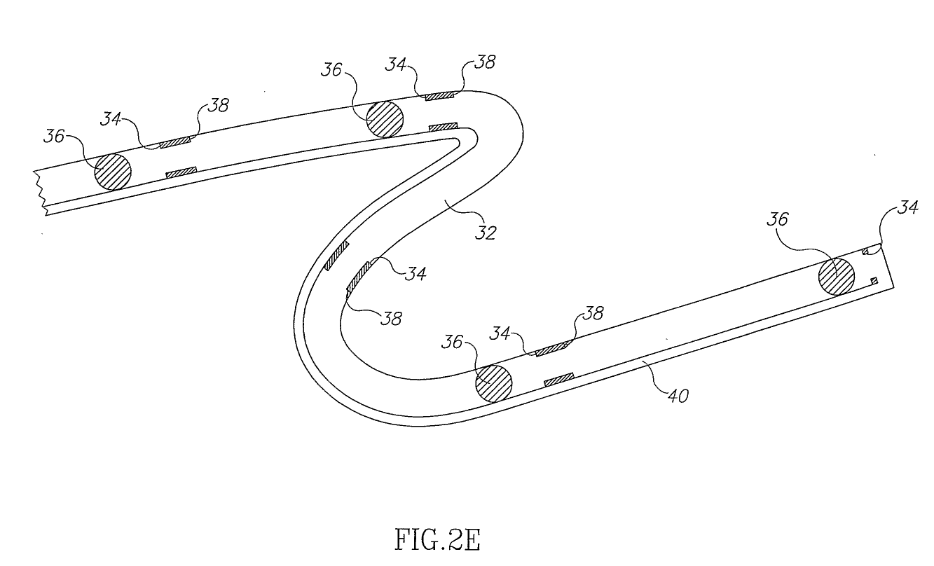

Reference is now made to FIGS. 1-2D, which illustrate a system 10, constructed and operative in accordance with a non-limiting embodiment of the present invention.

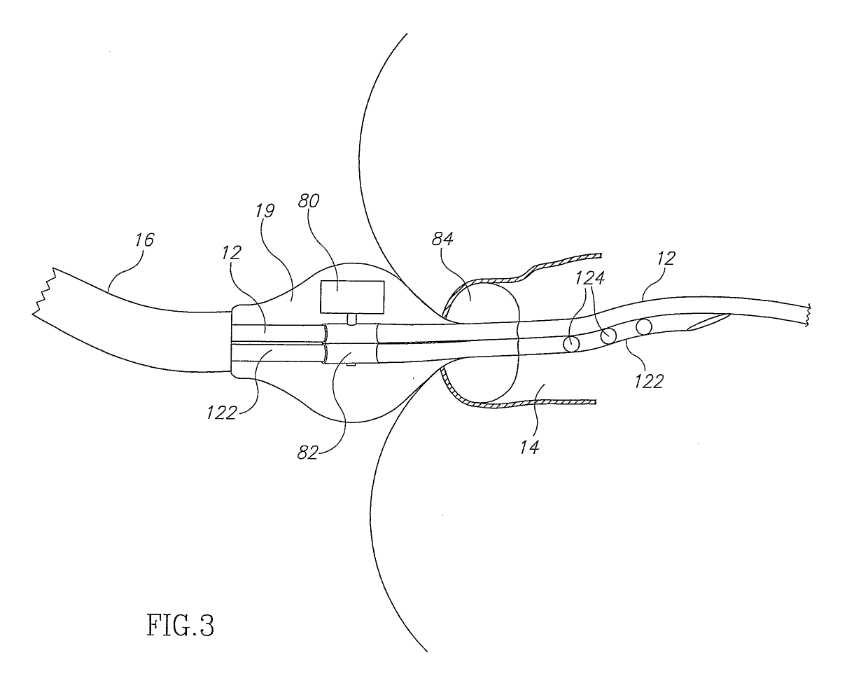

The system 10 includes a guide member 12 insertable into a body lumen 14 (e.g., the rectum and the GI tract), and constructed of any medically safe material. Guide member 12 may include a proximal flexible tube assembly 16 and a distal sheath 18 that covers the distal end of the tube of guide member 12. Both tube assembly 16 and sheath 18 are perhaps more rigid than the tube of guide member 12. (Proximal refers to the direction retracting away from the body lumen; distal is directed inwards and deeper into the body lumen.) A funnel introducer 19 (FIG. 1) may help insert the system 10 in the rectum and aid in sealing that end of the GI tract. Introducer 19 is described more in detail below with reference to FIG. 3.

A swivel head 23 is pivotally mounted (such as for omnidirectional swiveling) on the distal end of guide member 12, such as by means of a ball and socket joint 22. An imaging assembly 24 may be disposed in head 23, and may include, without limitation, an image sensor 26 (e.g., CCD or CMOS camera, or alternatively, x-ray, ultrasonic, MRI, infrared and/or microwave imaging devices) and light source 28 (e.g., a diode or fiber optics laser), all seen in FIG. 2B.

As seen best in FIGS. 2B and 2D, a lumen 30 may be formed through the swivel head 23 for passing therethrough tools for incision, biopsy sampling, etc. The tools may be introduced through a tube 31 that slips in lumen 30. When head 23 swings downwards in the send of FIG. 2D, lumen 30 moves leftward and tube 30 is situated more deeply into lumen 30. Conversely, when head 23 swings upwards in the send of FIG. 2D, lumen 30 moves rightward and tube 30 is situated less deeply into lumen 30. Other therapeutic or diagnostic devices may be introduced through lumen 30, as is practiced in colonoscopic or other endoscopic procedures.

Referring particularly to FIG. 2A, guide member 12 is formed with an internal channel 32 having a distal terminal end 34, which may be formed by a ring affixed at the end of channel 32. One or more percussion elements 36 (three are shown in the non-limiting illustrated embodiment) are disposed in channel 32. A proximal stop 38 may be stationed along a portion (or at the proximal end) of channel 32 distanced (near or far) from the distal end 34. As will be described below, percussion elements 36 can be forced distally along channel 32 to strike distal terminal end 34, thereby causing guide member 12 to advance distally in the body lumen 14.

It is noted that the term “percussion” throughout the specification and claims encompasses any kind of striking of two bodies against each other.

A second channel 40 is formed in guide member 12 in fluid communication with channel 32 at their distal ends. As will be described below, channel 40 is used to impart a force on percussion elements 36 that moves them proximally along channel 32 away from distal terminal end 34. (Alternatively, suction forces in channel 32 can be used to retract percussion elements 36 in channel 32 with no need for channel 40.) As will be described below, the percussion elements 36 can be moved proximally along channel 32 to strike proximal stop 38 to cause guide member 12 to retract proximally in the body lumen 14.

Head 23 may be swiveled and manipulated about joint 22 by means of a manipulator rod 42 disposed in a third channel 44 formed in guide member 12. As will be described below, fluid pressure may be introduced into channel 44 to push manipulator rod 42 distally against head 23. In the illustrated embodiment, three such channels 44 are provided, which are spaced 120° apart (FIG. 2C), so that head 23 and its imaging assembly 24 may be manipulated in any desired direction and orientation. Manipulator rod 42 may be spring-biased by a biasing device 46 (e.g., coil spring) to move proximally backwards. Wiring to the components of imaging assembly 24 may pass through another lumen 45 (shown in FIG. 2C), or alternatively may pass through other lumens (the ones shown and described above or additional lumens).

It is noted that manipulator rod 42 is just one example of a device for manipulating the swivel head 23 and many other devices can be used, such as but not limited to, inflatable balloons, pull/push wires, hydraulic/pneumatic devices, electrical devices such as servomotors, linear actuators, etc.

Alternatively, the electrical and optical components of imaging assembly 24 may have their own internal power source, with no need for external wiring. The imaging assembly 24 may wirelessly transmit or receive data to or from an external processor (not shown). The components of system 10 may be fully automated with sensors and operate in a closed or open control loop.

One of the lumens (e.g., 30) may be connected to a fluid source (not shown), e.g., pressurized water or air, for cleaning the area near imaging assembly 24 or for cleaning the body lumen 14 itself (e.g., the colon) or for inflating the body lumen 14. In this manner, the invention can perform cleansing of the GI tract prior to examination thereof in a colonoscopic procedure, and/or can clean the GI tract during the colonoscopic procedure. In this manner the patient may not need to go through any preparation procedure for cleaning the colon, such as an enema, prior to the colonoscopic procedure. During the colonoscopic procedure, following the insertion of guide member 12 into body lumen 14, forward propulsion forces together with pressurized cleansing liquid, e.g. water, will break down and dissolve solid waste as guide member 12 advances through body lumen 14. Forward movement forces may include forces applied both in the distal and proximal ends of the device, and may also include manual forces applied by the practitioner, and/or propulsion forces caused by percussion strikes, vibrations, water jets pulsation, or any combination thereof. All of these forces may assist guide member 12 in breaking and dissolving any solid waste within body lumen 14. Cleansing liquids and waste materials may be removed from body lumen 14 via a drain tube 122 as described below. The waste removal and draining may be carried out at any location along the lumen from the distal end to the proximal end (the lumen being provided with suitable drainage openings along its length).

Thus, the present invention can reduce or even obviate the need for aggravating pre-cleaning procedures widely used for preparing for a colonoscopy—no need for enemas, foul tasting substances that cause diarrhea for bowel preparation, strict liquid diet, etc. Rather the invention starts cleaning the colon upon entering the anus without causing discomfort to the patient. Imaging may be performed while passing the instrument into the colon or while extracting the instrument from the colon.

The following is a summary of a typical, but non-limiting, procedure of using system 10. First, the patient may either skip any pre-cleaning of the colon altogether or may undergo a much shorter and friendlier pre-cleaning, such as a small dosage of an enema (or a small amount of solution or a pill that causes diarrhea). Next, the system (also referred to as endoscopic device) 10 is inserted in the colon. Fluid is introduced in the endoscopic device 10 and the jet of fluid starts to clean the path for the endoscope in the colon. The endoscope may take images of the colon as the endoscope enters the colon and/or as the endoscope exits the colon. Any waste material may be collected in a waste container. After imaging and/or other procedures performed with tools through the working channel, the endoscope with endoscopic device 10 is removed from the colon. Any waste material collected during the colonoscopy is sealed in a waste container and disposed.

Reference is now made to FIG. 2E, which illustrates another embodiment in which guide member 12 includes multiple percussion elements 36 that are limited to travel in discrete regions of channel 32. At each discrete region, the percussion elements 36 can strike either a distal terminal end 34 or a proximal stop 38. The pressurized fluid that drives the percussion elements 36 can pass through one region to the other (that is, they are not sealed).

In the embodiment of FIG. 2E, the system is propelled by multiple local striking forces distributed along the guide member. The distribution of multi-propulsion forces along the length of the guide member enables propelling very long and twisted guide members (differently shaped and sized) along and through very long and twisted_body lumens and passages. Such an embodiment may be used for reaching far regions of the small intestine and for navigating through tight bends, for example.

Reference is now made to FIG. 3. Guide member 12 may be fed into the body lumen 14 by means of a tube mover 80 located in introducer 19 (or alternatively in force applicator 50 described below). Tube mover 80 may be a gear motor that drives one or more drive wheels 82 that frictionally cause guide member 12 to advance or retract. It is appreciated that this is just one example of a tube mover and many other devices can be used to advance or retract guide member 12. It is also noted that the portion of guide member 12 closest to the inlet of body lumen 14 (e.g., the rectum) may be simply manually manipulated (with little force), and that combined with the percussive forces of the percussion elements 36 advance or retract the guide member 12 in the body lumen 14. A seal 84 may be provided for sealing the system at the entrance to body lumen 14. Seal 84 may be, for example, a soft, squeezable seal or an inflatable seal.

It is further noted that instead of percussion elements, one or more vibratory elements may be positioned at a distal portion of guide member 12 or distributed along guide member 12. The vibration of guide member 12 or the distal portion of guide member 12 plus manual manipulation movement at the inlet of body lumen 14 can be used to advance or retract guide member 12 in the body lumen 14.

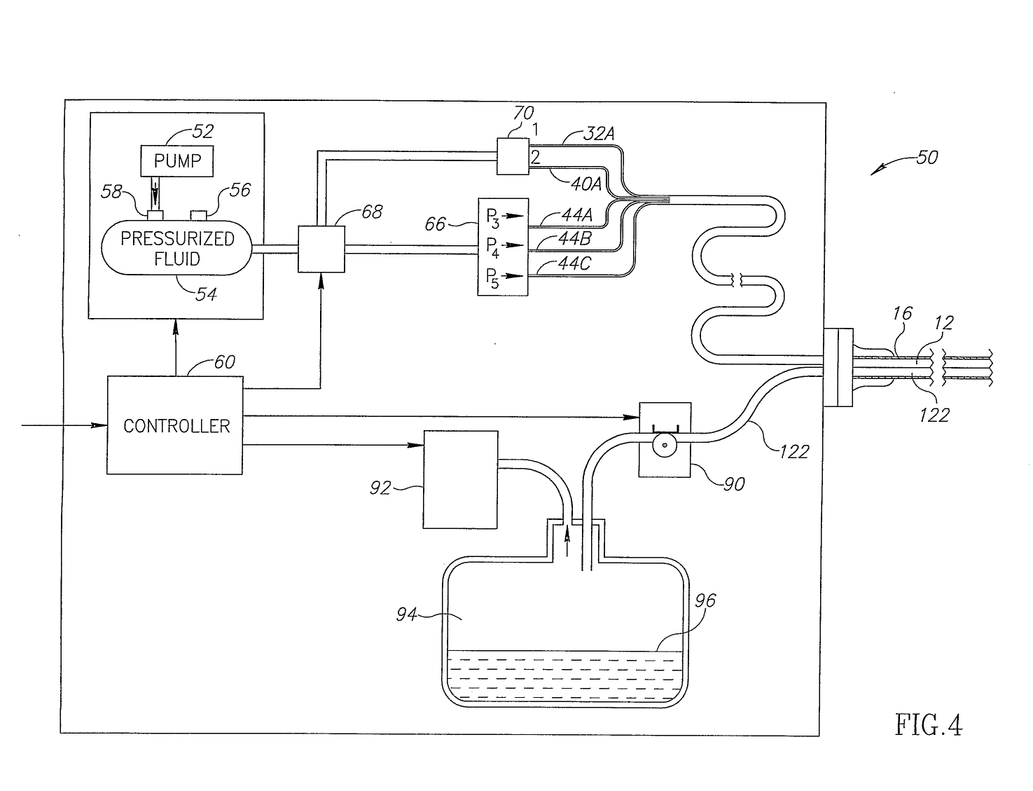

The system of the invention may be capable of draining fluids from the body lumen. Accordingly, a drain tube 122 may also be introduced into body lumen 14. Drain tube 122 may have apertures 124 formed along an axial length thereof to help fluids and waste materials enter therein. Drain tube 122 may be static or may be moved by tube mover 80. Referring to FIG. 4, it is seen that drain tube 122 may lead waste material via a pressure regulator 90 into a collection tank 94, in which drained material 96 may collect. A suction pump 92 may be connected to collection tank 94 to create negative pressure therein in order to suck the material from drain tube 122.

A force applicator 50 (FIG. 1) is provided that applies a force on the percussion elements 36 to propel them distally along channel 32 to strike terminal end 34 to advance guide member 12 distally in body lumen 14. Force applicator 50 is now described in detail with reference to FIG. 4. In the non-limiting illustrated embodiment, force applicator 50 includes a pump 52 that compresses fluid (preferably air, but could be CO₂, water or other liquids or gases) to a pressurized fluid reservoir 54. A pressure sensor 56 senses the pressure of reservoir 54 and a one-way check valve 58 connects reservoir 54 to pump 52. The operation of pump 52, as well as the rest of force applicator 50, is controlled by a controller 60. The operator can input instructions, data or any other information to controller 60 by means of a user interface unit 62 (FIG. 1). A display 64 (FIG. 1) may be provided to display any information related to the operation of the system 10 and the images and/or data acquired by the system of the invention. (The excess length of the tubing of guide member 12 may be located in force applicator, if desired.)

The pressurized fluid is directed to the various channels of system 10. For example, the pressurized fluid is directed (by suitable tubing/manifolds) to a control valve 66 that directs the pressurized fluid to three tubes 44A, 44B and 44C which are connected to the three channels 44 used to manipulate imaging assembly 24. The pressurized fluid is also directed, such as by means of a pressure regulator 68, to a pressure switch 70 that alternatively directs the pressurized fluid to either channel 32, via a tube 32A, for advancing percussion elements 36 distally in channel 32, or to channel 40, via a tube 40A, for retracting percussion elements 36 proximally in channel 32. Pressure switch 70 is preferably capable of quickly switching between the two channels 32 and 40 to move the percussion elements 36 back and forth quickly. The percussion elements 36 can strike the distal terminal end 34 with a series of strikes to move the guide member distally, or alternatively, the percussion elements 36 can strike the proximal stop 38 with a series of strikes to move the guide member proximally. The pressure is switched and regulated between the two channels 32 and 40 so as to release any trapped air or other fluids and ensure smooth and flawless operation of the system.

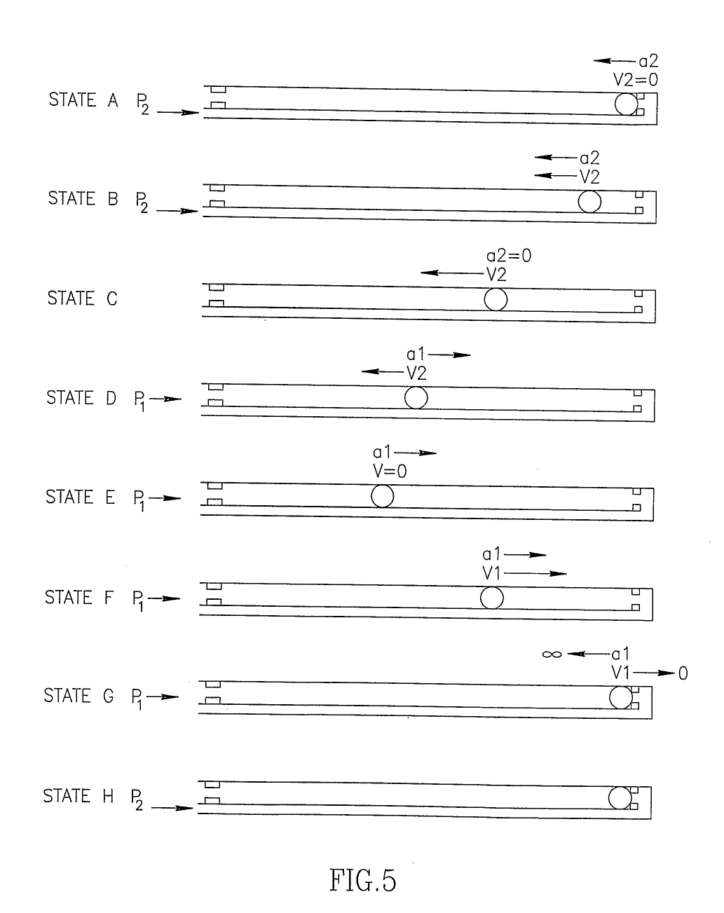

Thus, force applicator 50 can apply a sequence of forces on percussion elements 36, as described further below with reference to FIG. 5. The sequence of forces includes a first force P1 that propels the percussion element 36 distally along channel 32 towards terminal end 34, and a second force P2 that retracts percussion element 36 proximally away from terminal end 34.

The sequence of forces applied by force applicator 50 is schematically shown in FIG. 5 (it is emphasized that these are merely examples and the invention is not limited to these examples). In state A, percussion element 36 has traveled distally along channel 32 and is at terminal end 34. At this point, force P2 is applied via channel 40 that accelerates percussion element 36 proximally.

In state B, continued application of force P2 moves percussion element 36 proximally along channel 32 with acceleration a2, which causes percussion element 36 to move with increasing velocity v2. In state C, controller 60 (FIG. 4) ceases application of force P2 on percussion element 36. The percussion element 36 continues to move proximally along channel 32 with velocity v2, but the acceleration a2 is now zero.

In state D, force P1 is applied via channel 32 that distally accelerates percussion element 36 at acceleration a1. In state E, the velocity of percussion element 36 changes from the proximally directed velocity v2 to zero. In state F, continued application of force P1 moves percussion element 36 distally at velocity v1 and acceleration a1. In state G, percussion element 36 impacts terminal end 34, thereby advancing guide member 12 distally in body lumen 14. State H shows percussion element 36 at rest at the terminal end 34 (state A) and the cycle (sequence) repeats itself.

In the above sequence, the percussion element 36 does not move all the way back to proximal stop 38, but rather each time moves proximally back a little before striking distal terminal end 34. In this manner, guide member 12 advances distally in body lumen 14. The sequence of forces can operate at a frequency of 0.1-50 Hz or more, for example.

In order to remove system 10 from the body lumen 14, system 10 may be simply pulled out of body lumen 14. Alternatively, a reverse sequence of movements of percussion element 36 can be used, wherein the percussion element 36 is forced to strike the proximal stop 38 and each time is moved distally a little (not striking terminal end 34) and then moved back proximally to strike proximal stop 38 again. In this manner, guide member 12 is retracted proximally from body lumen 14. At the same time, guide member 12 may be pulled backwards manually or by means of tube mover 80 and drive wheels 82.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A system for advancing in a body lumen comprising:
a guide member insertable into a body lumen;
apparatus for providing a forward propulsion force on said guide member to advance said guide member distally into said body lumen; and
a lumen formed through said guide member connected to a fluid source, wherein fluid flowing through and out of said lumen cleans said body lumen.

2. The system according to claim 1, wherein the fluid flows in said lumen and cleans said body lumen while the forward propulsion force propels said guide member distally in said body lumen.

3. The system according to claim 1, wherein the fluid flows in said lumen and cleans said body lumen prior to the forward propulsion force propelling said guide member distally in said body lumen.

4. The system according to claim 1, further comprising an imaging assembly disposed in a distal portion of said guide member adapted to create images of internal portions of said body lumen.

5. The system according to claim 1, further comprising a drain tube through which waste material from the fluid flowing in said body lumen is drained out of said body lumen.

6. The system according to claim 1, wherein the forward propulsion force together with said fluid breaks down and dissolves waste material as said guide member advances through said body lumen.

7. The system according to claim 1, wherein the apparatus for providing the forward propulsion force comprises at least one percussion element disposed in a first channel formed in said guide member, said first channel having a distal terminal end, wherein said at least one percussion element is arranged to be forced distally along said first channel to strike said distal terminal end, thereby causing said guide member to advance distally in the body lumen.

8. The system according to claim 1, wherein the apparatus for providing the forward propulsion force comprises a vibratory element that sets up vibrations in said guide member.

9. The system according to claim 1, wherein the apparatus for providing the forward propulsion force comprises a jet pulsation device that creates jet pulses of fluid to propel said guide member.

10. The system according to claim 4, wherein said imaging assembly is disposed in a swivel head pivotally mounted on a distal end of said guide member.

11. The system according to claim 10, wherein a lumen is formed through said swivel head for passing therethrough devices.

12. The system according to claim 10, wherein said head is swiveled and manipulated about a joint by means of a manipulator rod disposed in a third channel formed in said guide member.

13. The system according to claim 7, wherein a second channel is formed in said guide member in fluid communication with said first channel, said second channel being used to impart a force on said at least one percussion element that moves it proximally along said first channel away from said distal terminal end.

14. The system according to claim 7, wherein said guide member comprises multiple percussion elements that are limited to travel in discrete regions of said first channel, wherein at each discrete region, said percussion elements are arranged to strike either a distal terminal end or a proximal stop formed in said first channel for each discrete region.

15. A system for advancing in a body lumen comprising:
a guide member insertable into a body lumen;
an imaging assembly disposed in a distal portion of said guide member adapted to create images of internal portions of said body lumen;
at least one percussion element disposed in a first channel formed in said guide member, said first channel having a distal terminal end; and
a force applicator arranged to apply a force on said at least one percussion element to propel said at least one percussion element distally along said first channel and strike said distal terminal end, thereby causing said guide member to advance distally in the body lumen.

16. The system according to claim 15, wherein a second channel is formed in said guide member in fluid communication with said first channel, said second channel being used to impart a force on said at least one percussion element that moves it proximally along said first channel away from said distal terminal end.
